(19) United States
(12) Patent Application Publication
(10) Pub. No.: US 2026/0053340 A1
(43) Pub. Date: Feb. 26, 2026

(54) PATIENT GUIDANCE USING VISIBLE TARGET AND FEEDBACK

(71) Applicant: ALCON INC., Fribourg (CH)

(72) Inventor: Peter Zieger, Berlin (DE)

(21) Appl. No.: 19/296,155

(22) Filed: Aug. 11, 2025

Related U.S. Application Data
(60) Provisional application No. 63/686,921, filed on Aug. 26, 2024.

(51) Int. Cl. A61B 3/00; A61B 3/032; A61B 3/113; A61B 3/14

(52) U.S. Cl. CPC A61B 3/0041; A61B 3/0091; A61B 3/032; A61B 3/113; A61B 3/14

(57) ABSTRACT

A system for guiding a patient during an ophthalmic examination includes a display screen and a patient guidance system (PGS). The PGS receives an enabling signal that identifies an examination device to be used during the examination. In response to receiving the enabling signal, the PGS may display a visible target via the display screen and/or play an audible sound via a speaker. The audio sound is a clear indication of the examination sequence performed using the examination device. The visible target includes patient instructions corresponding to an examination sequence performed using the examination device. The PGS automatically modifies a characteristic of the visible target or audio signal when the patient does not comply with the patient instructions during the examination sequence.

### INTRODUCTION

The present disclosure relates to hardware and associated software-based solutions for automated interaction with a patient during an ophthalmic procedure, e.g., a preoperative or in-office eye examination.

Diagnostic devices used in the field of ophthalmology typically require a patient to look directly into the device so that the device is able to measure internal or external parameters of the eye. Each device is configured to perform a specific measurement in a precise manner, and thus typically requires an expected patient posture and eye position for proper measurement accuracy and repeatability. In practice, ocular measurements frequently suffer from unexpected patient head or eye motion. This requires the operator of the device to repeat the required measurements, which in turn extends the duration of the procedure while reducing patient comfort and satisfaction.

### SUMMARY

Disclosed herein are an automated system and associated computer-based methodology for interacting with a patient during an ophthalmic procedure, exemplified herein as an in-office eye examination. The present disclosure enables implementation of a real-time, technician-monitored solution for controlling a visible target to actively guide the patient through a device-specific examination sequence, i.e., a series of required patient actions. Among other attendant benefits, the present solutions reduce the duration of a given eye examination while enabling accurate and repeatable collection of optical measurements. The solutions described herein are ultimately geared toward effectively and efficiently informing a clinician as to possible treatment options while improving the overall patient experience.

As noted above, unsatisfactory optical measurements can result from unexpected motion of a patient's head or eyes, including blinking or looking away from a device or other commanded focal point during the course of the examination. Within a typical examination room, an operator of a measurement device may not always communicate in a sufficiently clear or articulate manner for a given patient. The patient's age, visual acuity, or hearing loss may likewise complicate the examination process, thus leading to potential measurement errors. The operator and patient may also speak different languages. Any of these example scenarios can lead to increased time in the examination chair, patient dissatisfaction, and suboptimal examination results. Additionally, verbal commands such as “keep still”, “open your eyes wide”, “please blink”, etc., tend to be indefinite or vague, thus leading to possible confusion and patient-to-patient response variation. The present automated approach is therefore intended to improve upon the current state of the art in terms of patient satisfaction and ophthalmic examination results.

In particular, a system is disclosed herein for guiding a patient during an ophthalmic examination. A representative embodiment of the system includes a display screen and a computer-based hardware and software system, the latter of which is referred to herein as a patient guidance system (PGS). The PGS is in communication with the display screen and includes a processor. The processor is configured to receive an enabling signal, e.g., from a user interface device, that identifies an examination device to be used during the ophthalmic examination. In response to receiving the enabling signal, the PGS/processor displays a visible target via the display screen. The visible target includes patient instructions corresponding to an examination sequence to be performed by the patient during use of the examination device. As part of its programmed functionality, the PGS, via its processor, automatically modifies a characteristic of the visible target when the patient does not comply with the patient instructions during the examination sequence.

The visible target may include a fixation target, e.g., a message constructed of alphanumeric characters or graphical symbols/images. In other embodiments, the visible target may include a fogging target. In the case of the fogging target, the enabling signal may identify a refractive error of the patient. The PGS may then automatically adjust a characteristic of the fogging target in response to the enabling signal.

One or more sensors, e.g., a camera such as an infrared gaze tracking camera, may be configured to detect an eye position of the patient, and to transmit a feedback signal to the PGS indicative of the eye position. The PGS in such an embodiment is configured to detect, using the feedback signal, a noncompliant condition during which the patient does not comply with the patient instructions.

In one or more embodiments, the enabling signal identifies a preferred language of the patient. The PGS in such embodiments is configured to display the visible patient instructions in the preferred language of the patient in response to the enabling signal.

The system may optionally include one or more speakers. The enabling signal may include a request for audible patient instructions. The PGS in such an implementation is configured, in response to receiving the enabling signal, for simultaneously broadcasting the audible patient instructions while displaying the visible patient instructions.

Another aspect of the disclosure pertains to a method for guiding a patient during an ophthalmic examination. The method in accordance with a possible implementation includes receiving, via the PGS, an enabling signal that identifies an examination device to be used during the ophthalmic examination. In response to receiving the enabling signal, the method includes displaying a visible target via the display screen, with the visible target including patient instructions corresponding to an examination sequence performed using the examination device. The method also includes automatically modifying a characteristic of the visible target when the patient does not comply with the visible patient instructions during the examination sequence.

The above-summarized system in accordance with another embodiment includes a gaze tracking camera, a display screen, a user interface device operable for generating an enabling signal, and a patient guidance system (PGS) in communication with the gaze tracking camera and the display screen. The PGS in this representative construction is configured to receive an enabling signal that identifies an examination device to be used during the ophthalmic examination. In response to receiving the enabling signal, the PGS displays a visible target via the display screen, the visible target including patient instructions corresponding to an examination sequence to be performed using the examination device. The PGS also receives a feedback signal from the gaze tracking camera indicative of an eye position of the patient and automatically modifies a characteristic of the visible target when, based on the eye position of the patient, the patient does not comply with the visible patient instructions during the examination sequence. The visible target in this particular implementation includes at least a fixation target comprised of alphanumeric characters, graphical symbols, or images.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 is an illustration of an automated patient guidance system (PGS) constructed in accordance with the present disclosure.

FIG. 2 depicts a representative fixation target that may be controlled via the system illustrated in FIG. 1 as part of the present approach.

FIG. 3 illustrates an alphanumeric guidance sequence that may be presented via the system of FIG. 1 during an eye examination.

FIG. 4 illustrates a representative fogging target that may be used in one or more implementations of the alphanumeric guidance sequence shown in FIG. 4.

FIG. 5 illustrates a graphical guidance sequence of FIG. 1 that may be used as an alternative to or in conjunction with the guidance sequence of FIG. 3.

FIG. 6 is a flow chart describing a patient guidance method in accordance with a possible implementation.

The appended drawings are not necessarily to scale, and may present a somewhat simplified representation of various features of the present disclosure, including, for example, specific dimensions, orientations, locations, and shapes. Details associated with such features will be determined in part by the particular intended application and use environment.

### DETAILED DESCRIPTION

The present disclosure is susceptible of embodiment in many different forms. Representative examples of the disclosure are shown in the drawings and described herein in detail as non-limiting examples of the disclosed principles. To that end, elements and limitations described in the Abstract, Introduction, Summary, and Detailed Description sections, but not explicitly set forth in the claims, should not be incorporated into the claims, singly or collectively, by implication, inference, or otherwise.

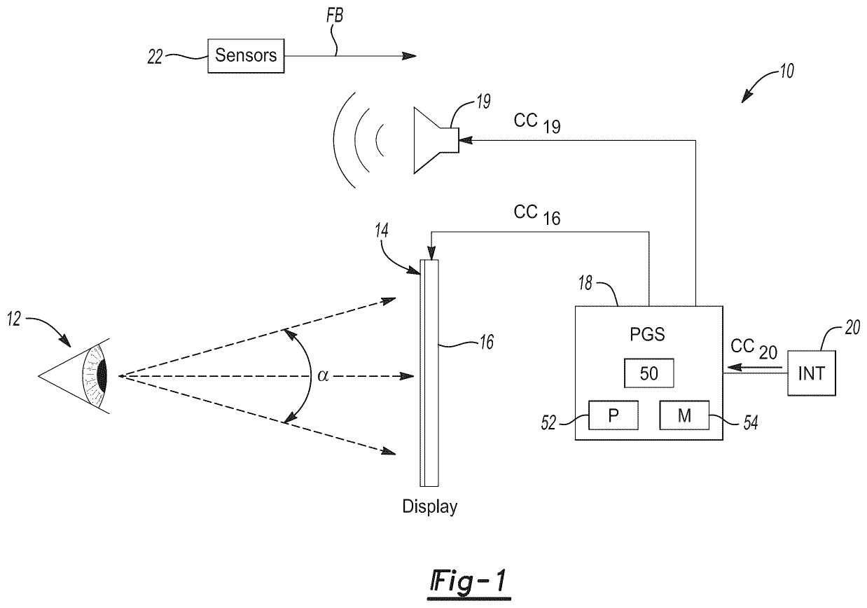

Referring to the drawings, wherein like reference numbers refer to like features throughout the several views, FIG. 1 depicts an automated system 10 for guiding a patient during an ophthalmic examination. An eye 12 of the patient is shown for illustrative simplicity. As the patient sits in an examination chair (not shown), the patient is required to view a visible target 14 on a display screen 16, e.g., a high-resolution light-emitting diode (LED), organic LED (OLED), liquid crystal display (LCD) with edge-lit LED backlights, etc.

As set forth herein, the position of the visible target 14 relative to the eye 12 and the alphanumeric and/or graphical content of the visible target 14 are selected and controlled by a patient guidance system (PGS) 18, with possible changes in eye position or line-of-sight of the eye 12 indicated in FIG. 1 by a viewing angle (α). As noted below, a technician or clinician may participate in the examination using a user interface device (INT) 20 in one or more embodiments, such as when selecting the procedure and identifying patient traits such as age, sex, height, refractive error, injury/disease history, and/or a preferred language of the patient.

The PGS 18 as contemplated herein is in communication with the display screen 16 and the user interface device 20, along with one or more sensors (“Sensors”) 22 as described in further detail below. The PGS 18 may be embodied as one or more computer devices operable for performing the various functions described herein. The PGS 18 may be generally composed of one or more processors (P) 24, e.g., a discrete microprocessor, application specific integrated circuit (ASIC), or dedicated control module. The PGS 18 also includes and computer-readable storage medium or media, i.e., memory (M) 25, components of which are non-transitory and non-volatile and thus configured to record thereon instructions embodying a method 50 (see FIG. 6). That is, aspects of the memory 25 may include a CD-ROM, magnetic disk, solid-state drive (SSD) memory, a hard-disk drive (HDD) memory, etc., along with any necessary transient or volatile memory, flash memory, random access memory, etc.

Aspects of the method 50 may be practiced with a wide variety of computer-network configurations, including but not limited to multiprocessor systems, local area networks, wide area networks, and the like. The method 50 may be practiced in distributed-computing environments where tasks are performed by resident and remote-processing devices that are linked through a communications network. In a distributed-computing environment, for instance, program modules may be located in both local and remote computer-storage media including memory storage devices. Aspects of the present disclosure may therefore be implemented in connection with various hardware, software, or a combination thereof, in a computer system or other processing system.

With respect to the system 10 of FIG. 1, the PGS 18 is configured to receive an enabling signal (CC₂₀) from a remote device, which in turn is generated by the user interface device 20 in the representative construction of FIG. 1. The enabling signal (CC₂₀) as contemplated herein is an electronic signal conveying information to the PGS 18 regarding the patient and the examination procedure, along with other possible information. In particular, the enabling signal (CC₂₀) identifies an examination device to be used during a given phase of the ophthalmic examination. The PGS 18 may be configured to display the visible patient instructions 14 in the preferred language of the patient in response to the enabling signal (CC₂₀) in one or more embodiments, as noted elsewhere hereinabove.

In a typical eye examination, for instance, such devices may include something as simple as a displayed Snellen chart, in which case the “device” includes the visible target 14 itself. When performing measurements of the eye 12, the device may include a phoropter, a corneal topographer, a tonometer, an auto refactor, a slit lamp, retinoscope, an ophthalmoscope, a visual field analyzer, or a variety of other possible devices. Each device may have a corresponding examination sequence requiring the patient to perform different actions or hold certain poses for a particular duration. For instance, the patient may be required to focus into the device and/or on the visible target 14 for a set period, e.g., five seconds, or to remain still, keep the eye 12 open (possibly to a particular level or for a specific duration), blink a certain number of times, etc.

In response to receiving the enabling signal (CC₂₀), the PGS 18 is configured to display the visible target 14 via the display screen 16 at a specific location thereon, for instance by transmitting a display control signal (CC₁₆) to a resident control circuit (not shown) of the display screen 16 to command the size, shape, color, and other content of the displayed information. The visible target 14 as contemplated herein includes visible patient instructions corresponding to an examination sequence to be performed using the identified examination device. Different examples of such information are described below with reference to FIGS. 3-5.

The PGS 18 of FIG. 1 is also configured or operable to automatically modify a characteristic of the visible target 14 when the patient does not comply with the visible patient instructions during the examination sequence. Thus, the present solution may entail the use of feedback signals (arrow FB) from the one or more sensors 22. Measurements from such sensors 22 may be used by the PGS 18 to detect a noncompliant condition or error. By way of a non-limiting example, the patient may not blink after being instructed to do so, or the patient may not hold open the eye 12 for a required duration. Alternatively, the patient's eye 12 may be held open, but perhaps not widely enough for accurate measurements to be collected. The patient may look down when instructed to look up. These and other instances of noncompliance may adversely affect the measurement results and require repeat measurements or inaccurate measurement results.

In order to detect characteristics of the eye 12 in the scope of the present disclosure, the sensor(s) 22 may include an imaging system such as a camera, an optical coherence tomography (OCT) machine, wavefront measurement, or any another device that is operable for determining the open/closed state of the eye 12, and possibly its viewing angle (α) relative to the visible target 14. Such a sensor 22 may include a charge-coupled device (CCD) or complementary metal-oxide semiconductor (CMOS) image sensor. Options include video-based or web-cam eye tracking cameras and associated image processing logic, or infrared (IR) gaze tracking cameras for tracking corneal reflections and pupil position in a three-dimensional space. Specific technologies such as pupil center corneal reflection (PCCR) gaze trackers may be used in a similar manner. The sensor(s) 22 may transmit the feedback signal (arrow FB) to the PGS 18 in real-time, with variations of measured eye state/position/gaze direction from an instructed state/position/gaze direction being indicative of the noncompliant condition. In the non-limiting example of the sensor(s) 22 including a gaze tracking camera, in other words, the feedback signal (arrow FB) is indicative of an eye position of the patient, i.e., the direction the patient is looking/line of sight.

The system 10 in one or more embodiments may therefore include the sensors 22 in the form of a gaze tracking camera, with the system 10 also including the display screen 16, the user interface device 20 operable for generating the enabling signal (CC₂₀), and the PGS 18. The PGS 18 is in communication with the gaze tracking camera and the display screen 16. The PGS 18 may be optionally configured to receive the enabling signal (CC₂₀) that identifies an examination device to be used during the ophthalmic examination. In response to receiving the enabling signal (CC₂₀), the PGS 18 in this embodiment may display the visible target 14 via the display screen 16, with the visible target 14 including patient instructions corresponding to an examination sequence to be performed using the examination device.

The PGS 18 also receives the feedback signal (FB) from the gaze tracking camera indicative of an eye position of the patient, and automatically modifies a characteristic of the visible target 14 when, based on the eye position of the patient, the patient does not comply with the visible patient instructions during the examination sequence. The visible target 14 may include at least the fixation target comprised of alphanumeric characters, graphical symbols, or images.

The system 10 in one or more embodiments may therefore include the sensors 22 in the form of a gaze tracking camera, with the system 10 also including the display screen 16, the user interface device 20 operable for generating the enabling signal (CC₂₀), and the PGS 18. The PGS 18 is in communication with the gaze tracking camera and the display screen 16. The PGS 18 may be optionally configured to receive the enabling signal (CC₂₀) that identifies an examination device to be used during the ophthalmic examination. In response to receiving the enabling signal (CC₂₀), the PGS 18 in this embodiment may display the visible target 14 via the display screen 16, with the visible target 14 including patient instructions corresponding to an examination sequence to be performed using the examination device. The PGS 18 also receives the feedback signal (FB) from the gaze tracking camera indicative of an eye position of the patient, and automatically modifies a characteristic of the visible target 14 when, based on the eye position of the patient, the patient does not comply with the visible patient instructions during the examination sequence. The visible target 14 may include at least the fixation target comprised of alphanumeric characters, graphical symbols, or images as described herein.

Within the scope of the present disclosure, the system 10 of FIG. 1 may be augmented with an optional speaker 19 controllable via audio control signals (CC₁₉) from the PGS 18, i.e., one or more audio speakers arranged in sufficiently close proximity to the patient such that the patient is able to clearly hear broadcasted instructions. In some implementations, the enabling signal (CC₂₀) may include a request for audible patient instructions, for example when the patient is visually impaired or would otherwise benefit from audible instructions. In such a case, the PGS 18 may be configured, in response to receiving the enabling signal (CC₂₀), to simultaneously broadcast the audible patient instructions while displaying of the visible instructions. For example, the patient may hear the utterance “open eyes wide” while simultaneously viewing the same message as text.

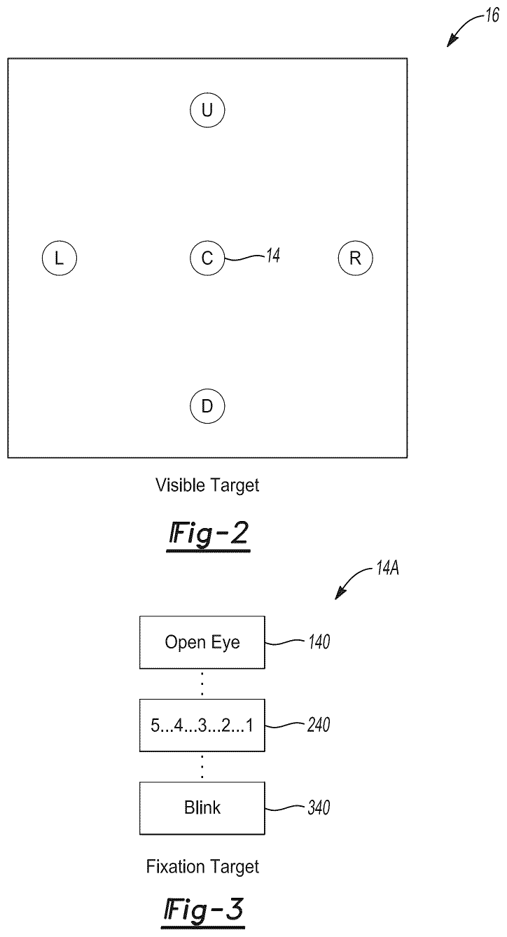

Referring briefly to FIG. 2, the visible target 14 is illustrated in various positions on the display screen 16. A center point (C) of the display screen 16 corresponds to a normal forward viewing angle (α) of FIG. 1. Thus, when the patient is instructed to “look straight ahead”, the eye 12 of FIG. 1 would focus on information displayed at the center point (C). The center point (C) is surrounded by various other focal points (U), (D), (L), and (R) corresponding to “up”, “down”, “left”, and “right”, respectively. In accordance with the disclosure, therefore, the visible patient instructions comprising the visible target 14 may be moved around the display screen 16 as needed by the PGS 18 of FIG. 1 in response to the enabling signal (CC₂₀) or overriding commands from the attending technician. The form and content of the visible target 14, including the number and/or location of displayed positions and associated dwell times, may vary within the scope of the disclosure, with non-limiting examples illustrated in FIGS. 3, 4, and 5.

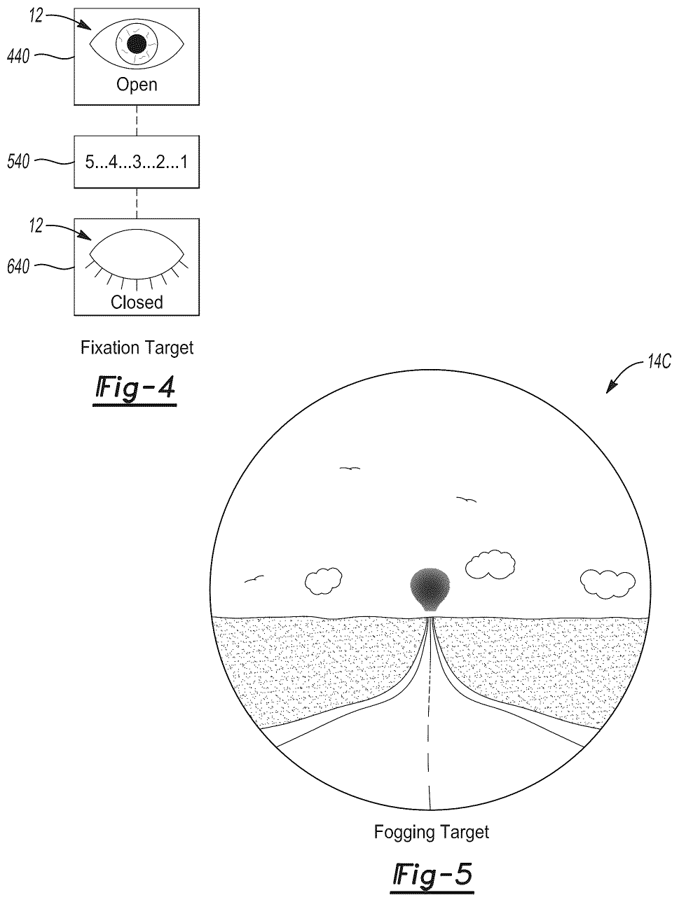

Referring now to FIGS. 3 and 4, the visible target 14 of FIGS. 1 and 2 may be optionally embodied as a fixation target 14A (FIG. 3) and 14B (FIG. 4). In FIG. 3, for instance, the fixation target 14A includes alphanumeric characters such as short phrases or numerical sequences. In such an implementation, the visible patient instructions contained in the fixation target 14A are presented as an examination sequence, represented as blocks 140, 240, and 340 in the non-limiting example of FIG. 3. Block 140 may convey a particular instruction, e.g., “Open Eye”, possibly followed by a timed count down at block 240, e.g., 5 . . . 4 . . . 3 . . . 2 . . . 1, followed in turn by another instruction such as “Blink” at block 340. As stated above, the text may be presented in the preferred language of the patient as initially communicated to the PGS 18 of FIG. 1 by the enabling signal (CC₂₀). Characteristics such as font type/size/color may also be varied as needed to customize the presented instructions to the patient's needs or preferences.

As shown in FIG. 4, the fixation target 14B may include one or more graphical symbols or images, either alone as shown or in conjunction with alphanumeric characters as depicted in FIG. 3. Using such symbols, the patient may be instructed as to required actions or poses in an intuitive manner. Use of the fixation target 14B may be advantageous in various circumstances, such as when the patient is a young child that may not yet be able to accurately read displayed phrases. In the non-limiting example of FIG. 4, for instance, block 440 illustrates a symbol of the eye 12 in an open state. This graphic may be followed by a countdown graphic at block 540 analogous to block 240 of FIG. 3. Block 540 in turn may be followed by display of another graphic, e.g., a symbol of the eye 12 in a closed state. In this example, the patient is therefore instructed to keep her eye 12 open for five seconds, with block 540 informing the patient as to the remaining amount of time for maintaining the open state. The visible instructions conveyed via the fixation targets 14A and 14B of FIGS. 3 and 4 may be accompanied by broadcast of audible patient instructions via the speaker(s) 19 of FIG. 1 as described above.

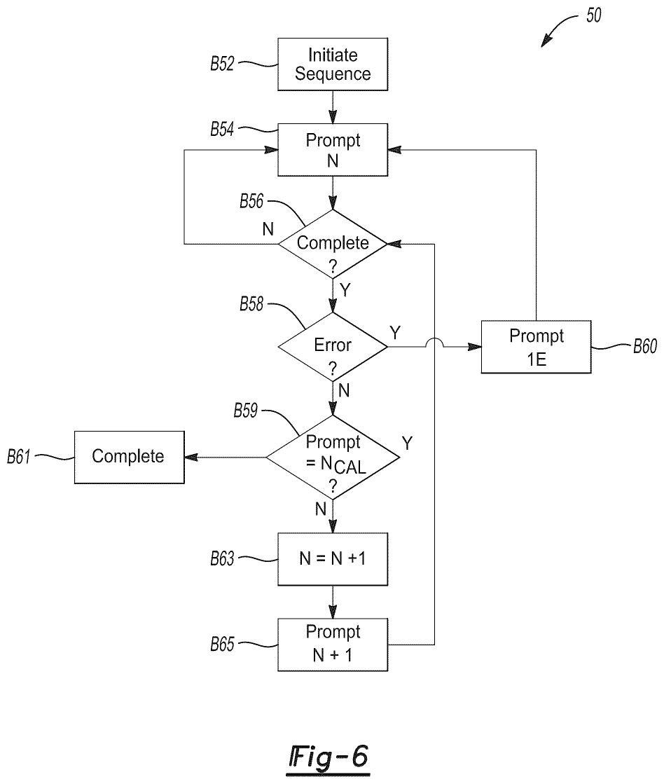

Referring briefly to FIG. 5, in one or more embodiments the visible target 14 of FIG. 1 may include a fogging target 14C. As appreciated in the art, fogging targets such as the representative fogging target 14C are sometimes used as visual stimuli during an eye examination or in conjunction with certain eye surgeries to relax ciliary muscles of the eye 12 and control its accommodation. As the name suggests, a “fogging” target is one that is slightly blurred or out of focus. This in turn helps prevent the eye 12 from actively focusing on the visible target 14 beyond maintaining a directed viewing angle (α). Use of the fogging target 14C can ensure accurate measurement of the eye's refractive error without the influence of accommodation, and may be helpful when using, e.g., autorefractors or wavefront aberrometers, among other devices.

In one or more embodiments, the PGS 18 of FIG. 1 may be configured to automatically adjust a blurring level of the fogging target 14C. This action may occur in response to the enabling signal (CC₂₀) of FIG. 1, for instance as a preset blurring level. Such a blurring level may correspond to a characteristic of the patient, such as the patient's visual acuity level or refractive error. Alternatively, the technician/clinician may update the blurring level as needed during the examination using the user interface device 20. In this embodiment and possibly other embodiments, the user interface device 20 may be variously configured as a touch screen device, a push-button device, a voice-activated system using speech recognition software, etc.

Referring to FIG. 6, instructions embodying the method 50 may be recorded in non-transitory/non-volatile portions of the memory 25 of FIG. 1 and executed by the processor 24 to perform the described functions. Constituent process steps of the method 50 are described in terms of logical code segments or functional blocks for illustrative clarity, with each block being executable by the processor 24 of FIG. 1 in the course of an eye examination or other ophthalmic procedure performed using the system 10 of FIG. 1.

In general, the method 50 is operable for guiding a patient during an ophthalmic examination, and includes receiving, via the PGS 18, the enabling signal (CC₂₀) that identifies an examination device to be used during the ophthalmic examination. In response to receiving the enabling signal (CC₂₀), the method 50 includes displaying the visible target 14 via the display screen 16, with the visible target 14 including patient instructions corresponding to an examination sequence performed using the examination device noted above. The method 50 also includes automatically modifying a characteristic of the visible target 14 when the patient does not comply with the visible patient instructions during the examination sequence.

A representative embodiment of the method 50 begins with block B52 (“Initiate Sequence”) with receipt of the enabling signal (CC₂₀) of FIG. 1 by the PGS 18. As described above, the enabling signal (CC₂₀) initiates the examination sequence by identifying a particular examination device to be used during the ophthalmic examination, along with providing other identifying information such as the patient's age, acuity, preferred language, and other characteristics. The method 50 proceeds to block B54 once the examination sequence has been completed.

Block B54 (“Prompt N”) is performed in response to receipt of the enabling signal (CC₂₀) by the PGS 18. Here, the PGS 18 displays a prompt in the form of the visible target 14 of FIG. 1 as one of the visible targets 14A, 14B, or 14C of FIGS. 3-5, respectively. The visible target 14 includes visible patient instructions corresponding to the examination sequence initiated at block B52. The method 50 thereafter proceeds to block B56.

At block B56 (“Complete?”) of FIG. 6, the method 50 includes determining if display of the sequence step presented to the patient in block B54 is complete before progressing to the next step in the examination sequence. The method 50 then proceeds to block B58.

At block B58 (“Error?”), the PGS 18 of FIG. 1 determines whether the patient properly complied with the display instructions from block B56. Embodiments of block B58 may entail the use of the sensor(s) 22 of FIG. 1 and the feedback signals (arrow FB) to determine compliance. For instance, if the instruction included “Open Eye”, block B58 may entail detecting, via the sensors 22, whether the patient did in fact open the eye 12. The method 50 then proceeds to block B59 when the PGS 18 determines that the patient properly complied, i.e., that an error was not detected. The method 50 proceeds in the alternative to block B60.

Block B59 (“Prompt N=N_{CAL}?”) includes determining via the PGS 18 whether the current prompt (N) from block B54 is the last prompt (N_{CAL}) in the examination sequence. Using the simplified three-step sequence of FIG. 3, for instance, block B59 would include determining if block 340 had been correctly completed, with block 340 being N_{CAL} in this example. The method 50 proceeds to block B61 when the PGS 18 determines or confirms that N=N_{CAL}, and to block B63 in the alternative when the prompt (N) of block B54 is not the last prompt (N_{CAL}).

Continuing with the discussion of FIG. 6, block B60 (“Prompt 1E”) may entail displaying an error message or corrective instruction to the patient via the display screen 16 of FIG. 1. An audible error tone such as a buzzer may be sounded in some embodiments to alert the patient to an incorrect response, or the displayed visible target 14 may be caused to temporarily blink to the same effect. The PGS 18 may be optionally programmed to allow this “reset” to occur a set number of times before halting the examination sequence and calling for manual intervention by the technician or clinician. The method 50 thereafter returns to block B54.

Block B61 (“Complete”) includes completing the examination sequence. Actions attendant to block B61 may include generating a diagnostic report based on the measurement results, and/or proceeding to another examination sequence. In the latter instance, the method 50 would commence anew from block B52 with initiation of a new sequence. At block B61, an audio response could be used to confirm that the measurement was successful.

Block B63 (“N=N+1”) includes incrementing the portion or stage of the examination sequence after successfully completing the prior stage at block B56, as verified at block B58. In keeping with the simplified three-step sequence of FIG. 3, for instance, block B63 could include loading sequence step 240 after successfully completing sequence step 140, or loading sequence step 340 after successfully completing sequence step 240. The method 50 thereafter proceeds to block B65.

Bock B65 (“Prompt N+1”) is analogous to block B54, and includes displaying one of the visible targets 14A, 14B, or 14C of FIGS. 3-5, respectively, corresponding to the particular sequence step N+1. The method 50 thereafter returns to block B56, with block B56 described above.

Embodiments of the present disclosure are described herein. The disclosed embodiments are merely examples, however, and thus other embodiments can take various and alternative forms. The drawings are not necessarily to scale. Some features could be exaggerated or minimized to show details of particular components. Therefore, specific structural and functional details disclosed herein are not to be interpreted as limiting, but merely as a representative basis for teaching one skilled in the art to variously employ the present disclosure.

Certain terminology may be used in the following description for the purpose of reference only, and thus are not intended to be limiting. For example, terms such as “above”and “below”refer to directions in the drawings to which reference is made. Terms such as “front”, “back”, “fore”, “aft”, “up”, “down”, “left”, “right”, “rear”, “side”, etc., describe the orientation and/or location of portions of the components or elements within a consistent but arbitrary frame of reference which is made clear by reference to the text and the associated drawings describing the components or elements under discussion. Moreover, terms such as “first”, “second”, “third”, and so on may be used to describe separate components. Such terminology may include the words specifically mentioned above, derivatives thereof, and words of similar import.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

We claim:

1. A system for guiding a patient during an ophthalmic examination, comprising:
a display screen; and
a patient guidance system (PGS) in communication with the display screen, wherein the PGS includes a processor configured to:
receive an enabling signal that identifies an examination device to be used during the ophthalmic examination;
in response to receiving the enabling signal, display a visible target via the display screen, the visible target including visible patient instructions corresponding to an examination sequence to be performed by the patient during use of the examination device; and
automatically modify a characteristic of the visible target when the patient does not comply with the visible patient instructions during the examination sequence.

2. The system of claim 1, wherein the visible target includes a fixation target.

3. The system of claim 2, wherein the fixation target is comprised of alphanumeric characters.

4. The system of claim 2, wherein the fixation target includes one or more graphical symbols or images.

5. The system of claim 1, wherein:
the visible target includes a fogging target; and
the PGS is configured to automatically adjust a characteristic of the fogging target in response to the enabling signal.

6. The system of claim 1, further comprising:
one or more sensors configured to detect an eye position of the patient and to transmit a feedback signal to the PGS indicative of the eye position, wherein the PGS is configured to detect, using the feedback signal, a noncompliant condition during which the patient does not comply with the visible patient instructions.

7. The system of claim 6, wherein the one or more sensors include a camera.

8. The system of claim 7, wherein the camera includes an infrared gaze tracking camera.

9. The system of claim 1, wherein the enabling signal identifies a preferred language of the patient, and wherein the PGS is configured to display the visible patient instructions in the preferred language of the patient in response to the enabling signal.

10. The system of claim 1, further comprising:
one or more speakers, wherein the enabling signal includes a request for audible patient instructions, and wherein the PGS is configured, in response to receiving the enabling signal, to simultaneously broadcast the audible patient instructions while displaying the visible patient instructions.

11. The system of claim 1, further comprising:
a user interface device operable for generating the enabling signal.

12. A method for guiding a patient during an ophthalmic examination, comprising:
receiving, via a patient guidance system (PGS), an enabling signal that identifies an examination device to be used during the ophthalmic examination;
in response to receiving the enabling signal, displaying a visible target via the display screen, the visible target including visible patient instructions corresponding to an examination sequence performed using the examination device; and
automatically modifying a characteristic of the visible target when the patient does not comply with the visible patient instructions during the examination sequence.

13. The method of claim 12, wherein displaying the visible target includes displaying a fixation target.

14. The method of claim 13, wherein displaying the fixation target includes displaying a message comprised of alphanumeric characters.

15. The method of claim 13, wherein displaying the fixation target includes displaying a message comprised of one or more graphical symbols or images.

16. The method of claim 12, wherein displaying the visible target includes displaying a fogging target, the method comprising:
automatically adjusting a characteristic of the fogging target in response to the enabling signal.

17. The method of claim 12, further comprising:
detecting, via the PGS using a feedback signal from one or more sensors, a noncompliant condition during which the patient does not comply with the visible patient instructions.

18. The method of claim 12, further comprising:
communicating a preferred language of the patient to the PGS via the enabling signal; and
displaying the visible patient instructions in the preferred language of the patient in response to the enabling signal.

19. The method of claim 12, further comprising:
simultaneously broadcasting audible patient instructions while displaying the visible patient instructions.

20. A system for guiding a patient during an ophthalmic examination, comprising:
a gaze tracking camera;
a display screen;
a user interface device operable for generating an enabling signal; and
a patient guidance system (PGS) in communication with the gaze tracking camera and the display screen, wherein the PGS is configured to:
receive an enabling signal that identifies an examination device to be used during the ophthalmic examination;
in response to receiving the enabling signal, display a visible target via the display screen, the visible target including visible patient instructions corresponding to an examination sequence to be performed using the examination device;
receive a feedback signal from the gaze tracking camera indicative of an eye position of the patient; and
automatically modify a characteristic of the visible target when, based on the eye position of the patient, the patient does not comply with the visible patient instructions during the examination sequence, wherein the visible target includes at least a fixation target comprised of alphanumeric characters, graphical symbols, or images.
